(19) United States
(12) Patent Application Publication
(10) Pub. No.: US 2016/0081668 A1
(43) Pub. Date: Mar. 24, 2016

(54) System and Method For Medical Imaging Calibration and Operation

(71) Applicant: The Johns Hopkins University, Baltimore, MD (US)

(72) Inventors: Gregory S. Chirikjian, Towson, MD (US); Martin K. Ackerman, Baltimore, MD (US); Emad M. Boctor, Baltimore, MD (US); Alexis Cheng, Baltimore, MD (US)

(21) Appl. No.: 14/470,606

(22) Filed: Aug. 27, 2014

Related U.S. Application Data
(60) Provisional application No. 61/870,385, filed on Aug. 27, 2013.

(51) Int. Cl. A61B 8/00; A61B 8/08; A61B 5/11; A61B 5/00; A61B 19/00

(52) U.S. Cl. CPC A61B 8/587; A61B 5/7271; A61B 5/725; A61B 5/742; A61B 19/5212; A61B 19/2203; A61B 5/1128; A61B 8/5215; A61B 2560/0223

(57) ABSTRACT

A system and method is provided for solving the AX=XB calibration problem. A calibration method is presented to determine the unknown X in the AX=XB calibration problem. Sensor data is filtered, such that data without the desired screw theory invariants are discarded. The correspondence between A and B is then computed, either through a probabilistic Batch method that treats the data streams as probability density functions, or by formulating the data streams as a time-evolving differential equation which allows for online calibration of the device. Also, a calibration phantom and software is also provided. The phantom is an extension of known Z-fiducial phantoms, in which the Z-fiducials are oriented based on consideration of imaging physics. An additional phantom is designed that does not utilize rods within the phantom to perform the calibration.

### CROSS-REFERENCE TO RELATED APPLICATIONS

This application is based on, claims priority to, and incorporates herein by reference U.S. Provisional Application Ser. No. 61/870,385, filed Aug. 27, 2013, and entitled “Apparatus and Method for the AX=XB Calibration Problem.”

### BACKGROUND

The present disclosure relates to medical imaging and, more particularly, to calibration of an ultrasound system or other imaging modality by solving for the calibration parameter, X, between A and B in the AX=XB calibration problem for noisy and temporally un-synced data.

Image-guided surgery systems are often used to provide surgeons with informational support. Due to several unique advantages, such as the absence of ionizing radiation, ease of use, and real-time imaging, ultrasound (US) is a common medical imaging modality used in image-guided surgery systems. To perform advanced forms of guidance with ultrasound, such as virtual image overlays or automated robotic actuation, an ultrasound calibration process must be performed. This process recovers the rigid body transformation between a tracked marker attached to the ultrasound transducer and the ultrasound image. A phantom or model with known geometry is also required.

There have been many different categories of phantoms or models used for ultrasound calibration including wall, cross-wire, Z-fiducial, and AX=XB phantoms. Z-fiducial phantoms are comprised of three wires, or rods, which are oriented in a plane to form a Z or an N shape. Given a plane intersecting all three wires of multiple Z-fiducials, the unique positioning of the plane can be determined. AX=XB phantoms are those that allow the relative rigid body transformation between two images to be recovered based on each image's content.

When utilizing AX=XB phantoms, a sensor calibration problem known as the AX=XB problem arises. In this problem, A, X, and B are homogeneous transformations, with A and B given from sensor measurements, and X is unknown. To solve for X, two compatible instances of independent exact measurements, (A¹, B¹) and (A², B²) can be used. However, sensor data often features asynchronous timing, differing sampling rates, dropped sensor readings, and/or noise that cause the relationship between A's and B's to be damaged.

It would therefore be desirable to have a sensor data collection apparatus (phantom) specifically designed to address constraints in ultrasound physics and image processing as well as a method to solve the AX=XB problem when the correspondence (temporal matching) between A's and B's is unknown and the data is noisy.

### SUMMARY OF THE INVENTION

The present disclosure overcomes the aforementioned drawbacks by providing both a method and an apparatus for solving the AX=XB calibration problem. A calibration method is presented to determine the unknown X in the AX=XB calibration problem. Sensor data is filtered, such that data without the desired screw theory invariants are discarded. The correspondence between A and B is then corrected, either through a probabilistic Batch method that treats the data streams as probability density functions (and therefore does not require correspondence), or by using the information given by the invariants to recreate the corresponded. The calibration parameter, X, can be found by solving the Batch equations or by formulating the data streams as a time-evolving differential equation which allows for online calibration of the device. Also, a calibration phantom and software is provided. The phantom is an extension of known Z-fiducial phantoms, in which the fiducials are oriented based on consideration of imaging physics. A further evolved extension of this phantom is also presented that does not utilize rod fiducials within the phantom to perform the calibration.

In one aspect of the disclosure, a system for calibrating an imaging system is described. The system includes an input to receive data acquired by at least one sensor of an imaging system, an input configured to receive data acquired by at least one sensor of a tracking system, and a processor to receive the data from the inputs. The processor uses the data to populate a calibration model having a form of AX=BX, wherein A, X, and B are homogeneous transformations with A and B determined from the data and X being an unknown calibration parameter, filters the data using screw theory invariants, computes the calibration parameter, X, between A and B, uses solutions from Batch methods which do not require correspondence of the input data, and finds correspondence using the invariants and finds the solution from a time-evolving method which allows real time computation and update.

Another aspect of the disclosure provides a phantom for calibration of an imaging system that includes a frame. The frame includes first and second opposing walls and a base with a first edge and a second edge. The first edge is joined to an edge of the first opposing wall and the second edge is joined to an edge of the second opposing wall. The phantom also includes a plurality of rods spaced between the first and second opposing walls. The rods lie in non-parallel planes.

In accordance with another aspect of the disclosure, a method is provided for calibration of an imaging system. The method includes providing a phantom and placing an imaging system in a first position relative to the phantom. The method also includes acquiring a first ultrasound image of the phantom, determining a spatial relationship between the phantom and the first image, and repositioning the imaging system in a second position relative to the phantom. The method further includes acquiring a second ultrasound image of the phantom, determining a second spatial relationship between the phantom and the second image, and relaying the first and second spatial relationships to a processor. The method also includes processing the spatial relationship data by using the data to populate a calibration model having a form of AX=BX, wherein A, X, and B are homogeneous transformations with A and B determined from the data and X being an unknown. Processing also includes filtering the data using a screw theory, disregarding portions of the data without desired screw theory invariants, and computing an unknown transformation between the imaging system and the phantom.

In accordance with yet another aspect of the disclosure, a phantom is provided for calibration of an imaging system. The phantom includes first and second opposing walls and a base with a first edge and a second edge. The first edge is joined to an edge of the first opposing wall and the second edge is joined to an edge of the second opposing wall. The phantom also includes a cover portion opposing the base, the cover portion having a first edge joined to an edge of the first opposing wall and a second edge joined to an edge of the second opposing wall and a plurality of apertures through the cover portion sized to receive a plurality of sensors.

The foregoing, and other aspects and advantages of the invention, will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a graphical representation of two rigid body motions modeled in accordance with the present disclosure.

FIG. 2 is a pair of graphs of shifted data streams for theta invariants and their correlation prepared in accordance with the present disclosure.

FIG. 3 is an illustrative representation of the calibration system in accordance with the present disclosure.

FIG. 4A is a representation of a previous calibration phantom model.

FIG. 4B is a representation of an updated calibration phantom.

FIG. 5 is a schematic illustration of a system and method of using the system in accordance with the present disclosure.

FIG. 6 is graphic illustration showing known points in a reference image and an affine transformation of these points, determined with image registration and/or tracking methods, to determine corresponding points in a new image.

### DETAILED DESCRIPTION OF THE INVENTION

The present disclosure provides a system and method for calibrating a medical imaging device. With the method of the present disclosure, calibration can be performed without knowing the correspondence between pairs of measurements that come from cameras, ultrasound probes, optical or magnetic pose tracking systems, and the like. Regardless of the specific device or constraints of a given modality, each imaging modality includes a “sensor” (be it, optical, magnetic, ultrasound, and the like) and, thus, the term “sensor” will be used herein to refer to a system or sub-system of a system that detects or collects data, such as cameras, ultrasound probes, optical or magnetic pose tracking systems, and the like.

The sensor calibration problem, AX=XB, appears often in the fields of robotics and computer vision. The variables A, X and B are rigid-body motions, with each pair of measurements (A, B) coming from sensors, and X is the unknown rigid-body motion that is found when solving the problem.

Any motion in Euclidean space can be described as a homogeneous transformation matrix of the form

$$H ( R , t ) = ( \begin{matrix}R & t \\ {0}^{T} & 1\end{matrix} ) \qquad ( 1 )$$

where t is a translation vector and R is a rotation matrix. In the three-dimensional case t∈ [symbol] ³ and R is 3×3 and is an element of the SO(3) group of orthogonal matrices. Given multiple equations of the form

AᵢX=XBᵢ [symbol] Aᵢ=XBᵢX⁻¹ (2)

a solution X∈SE(3) is sought in a wide variety of application.

A method is needed to solve for X wherein there does not need to be a priori knowledge of the correspondence between Aᵢ and Bᵢ. By viewing the two sets of reference frames Aᵢ and Bᵢ as probability densities ƒ_{A}H and ƒ_{B}H where H∈SE(3), and X as a probability density on SE(3) we can solve for X as the solution to a minimization problem where the cost is

C(X)=D_{KL}ƒ_{A}∥δ_{X}*ƒ_{B}*δ_{X-1} (3)

Where * denotes convolution on SE(3) and D_{KL}•∥• is the Kullback-Leibler divergence for SE(3).

The convolution of two probability density functions on SE(3) is defined as

ƒ₁*ƒ₂H′=∫_{SE(3)}ƒ₁Hƒ₂H⁻¹H′dH (4)

where H, H′∈SE(3) with H serving as a dummy variable of integration for each value of H′ and dH is the natural (bi-variant) Riemannian volume element for SE(3).

A Dirac delta function can be defined for SE(3) to have properties

∫_{SE(3)}δHdH=1 and ƒ*δH=ƒI₄

where [symbol] ₄=H( [symbol] ₃,0) is the 4×4 identity matrix which is the identity element of SE(3). Intuitively a Dirac delta can be though of as a function that has a spike with infinite height at the identity and vanishes everywhere else. A shifted Dirac delta function can be defined as

δ_{X}H=δX⁻¹H

which places the spike at X∈SE(3) and allows for equation (2) to be written as

δ_{A1}H=δ_{X}*δ_{B1}*δ_{X-1}H (5)

Because convolution is a linear operation on functions, we can write all n instances of 5) into a single equation of the form

$${f}_{A} H = {δ}_{X} * {f}_{B} * {δ}_{{X}^{- 1}} H \\ where \\ {f}_{A} H = \frac{1}{n} {\sum }_{i = 1}^{n} δ {A}_{i}^{- 1} H and {f}_{B} H = \frac{1}{n} {\sum }_{i = 1}^{n} δ {B}_{i}^{- 1} H . \qquad ( 6 )$$

When written in this form, it does not matter if the correspondence between Aᵢ and Bᵢ is known, as the above functions are normalized to be probability densities.

Functions ƒ_{A}(H) and ƒ_{B}(H) are not in L²(SE(3)), but this can be solved if each delta function can be replaced with a Gaussian distribution or the two whole set of delta functions, which result from samples obtained at discrete times, are each replaced with Gaussians that have the same mean and covariance at teach of the sets.

It is convenient to define the subset se_{<}(3)⊂se(3) which is a depleted version of SE(3) in which all screw rotations having an angle of π have been removed. The mean and covariance of a probability density ƒ(H) can be defined by the conditions

∫_{SE(3)} log M⁻¹HƒHdh=

[symbol]

and

Σ∫_{SE(3)} log [symbol] M⁻¹H[ log [symbol] M⁻¹H]^{T}ƒHdh. (7)

Explicit formulas defining the matrix exponential exp:se(3)→SE(3) and logarithm log:SE_{<}(3)→se(3) and log [symbol] :SE_{<}(3)∈ [symbol] ⁶ are reviewed below. The transpose on the second log log [symbol] H is defined to ensure that Σ is a 6×6 symmetric matrix. The operation log(H) takes any element in H∈SE_{<}(3) into the corresponding unique element in the Lie algebra SE(3) such that exp(log(H))=H. That is, the logarithm map is not surjective, unless we consider the target space to be se_{<}(3)⊂se(3), which can be though of as the Cartesian product of the open solid ball of radius π with [symbol] ³. Since SO(3) can be viewed as a solid closed three-dimensional ball of radius π with antipodal points identified, the exclusion of the bounding sphere of radius π in SE(3) defines a 5D set of measure zero that has no effect on the computation of Σ in the above equation.

We can therefore write

$$log H = ( \begin{matrix}Ω & v \\ {0}^{T} & 0\end{matrix} )$$

where Ω=−Ω^{T}∈so(3). The map V: se 3→ [symbol] ⁶ is then composed with the log to give z=log [symbol] H=[ωT,νT]∈ [symbol] ⁶ where ω∈ [symbol] ³ is the vector corresponding to Ω such that Ωx=ω×x for any x∈ [symbol] ³, where x is the vector cross product.

If ƒ(H) is a sum of Dirac deltas like ƒ_{A}(H) above, then

$${\sum }_{i = 1}^{n} log {M}_{A}^{- 1} {A}_{i} = 𝕆 \\ and \\ {\sum }_{A} = \frac{1}{n} {\sum }_{i = 1}^{n} {log}^{} {M}_{A}^{- 1} {{A}_{i} [ {log}^{} {M}_{A}^{- 1} {A}_{i} ]}^{T} \qquad ( 8 )$$

The adjoin matrix

$$Ad H = ( \begin{matrix}R & 𝕆 \\ tR & R\end{matrix} )$$

is used heavily in computations, as it has the property that

log [symbol] H₁H₂H₁⁻¹=AdH₁ log [symbol] H₂.

Here for any a∈ [symbol] ³, â is the skew-symmetric matrix such that ab=a×b. [symbol] is used as the reverse map which gives â [symbol] =a.

Evaluating the mean and covariance defined in (7) with the functions in (6) using the bi-invariance of the integration measure gives

M_{A}=XM_{B}X⁻¹ (9)

and

Σ_{A}=ADXΣ_{B}Ad^{T}X (10)

Taking the trace of both sides of (9) gives that the angle of rotation around the screw axes of M_{A} and M_{B} must be the same, θ_{A}=θ_{B}. This is one of the two invariants for SE(3).

To begin to solve for X, (9) can be rewritten as

log [symbol] M_{A}=ADX log [symbol] M_{B} (11)

in which the solution space of all possible X's that satisfy this equation is known to be two dimensional when the rotation angle is in the range (0, π). This can be seen by defining

$${log}^{} {M}_{A} = ( \begin{matrix}{θ}_{A} {n}_{A} \\ {v}_{A}\end{matrix} )$$

and breaking (11) up into rotational and translational parts as

n_{A}=R_{X}n_{B} (12)

and

V_{A}=θ_{B}t_{X}R_{X}n_{B}+R_{X}v_{B} (13)

Equation (12) has a one-dimensional solution space of the form R_{X}=Rn_{A},n_{B}Rn_{B}, φ where φ∈0,2π is free and Rn_{A},n_{B} is any rotation matrix that rotates the vector n_{B} into n_{A}. In particular, we choose

$$R {n}_{A} , {n}_{B} = 𝕀 + {n}_{B} \times {n}_{A} + \frac{1 - {n}_{B} \cdot {n}_{A}}{{‖ {n}_{B} \times {n}_{A} ‖}^{2}} {n}_{B} \times {{n}_{A}}^{2} \qquad ( 14 )$$

The rotation Rn_{B},φ is given by Euler's formula

Rn_{B},φ= [symbol] +sin φn_{B}+1−cos φn_{B}².

Substituting Rₓ=Rn_{A},n_{B}Rn_{B},φ into (13) gives

$$\frac{R {n}_{A} , {n}_{B} R {n}_{B} , φ {v}_{B} - {v}_{A} }{{θ}_{B}} = {n}_{A} {t}_{X} \qquad ( 15 )$$

where the skew-symmetric matrix n_{A} has rank 2, and a free translation degree of freedom exists in tₓ along the n_{A} direction. This means that tₓ can be defined as

t_{X}=t(s)=sn_{A}+am_{A}+bm_{A}×n_{A} (16)

where s∈ [symbol] is a second free parameter, m_{A} and m_{A}×n_{A} are defined to be orthogonal to n_{A} by construction. If n_{A}=[n₁,n₂,n₃]^{T} and n₁,n₂ are not simultaneously zero, then we define

$${m}_{A} ≐ \frac{1}{\sqrt{{n}_{1}^{2} + {n}_{2}^{2}}} ( \begin{matrix}- {n}_{2} \\ {n}_{1} \\ 0\end{matrix} ) .$$

The coefficients a and b are then computed by substituting (16) into (15) and using the fact that n_{A},m_{A},n_{A}×m_{A} is an orthonormal basis for [symbol] ³. Explicitly,

$$a = ( \frac{R {n}_{A} , {n}_{B} R {n}_{B} , φ {v}_{B} - {v}_{A} }{{θ}_{B}} ) \cdot {m}_{A}$$

$$and$$

$$b = ( \frac{R {n}_{A} , {n}_{B} R {n}_{B} , φ {v}_{B} - {v}_{A} }{{θ}_{B}} ) \cdot {m}_{A} \times {n}_{A} .$$

The feasible solutions can therefore be completely parameterized as

Xφ,s=HRn_{A},n_{B}Rn_{B},φ,t(s) (17)

where φ,s∈0,2π× [symbol] and H(R,t) is the same as in (1).

Given that (9) constrains the possible solutions, X(Ø, s), to a two-dimensional ‘cylinder’ defined by (17), solving for X reduces to that of solving (10) on this cylinder by calculating the values (Ø, s).

In one approach, we backsubstitute X=X(Ø, s) into (10) and minimize the cost function

C₁φ,s=∥Ad([Xφ,s]⁻¹)Σ_{A}−Σ_{B}Ad^{T}Xφ,s∥² (18)

The parameter s then appears linearly inside the norm, and C₁φ,s is quadratic in s and can be written as

C₁φ,s=C₁₀+C₁₁(φ)s+½C₁₂(φ)s².

The minimization over s can then be solved in closed form as

$$s = - \frac{{C}_{11} ( φ )}{{C}_{12} ( φ )} .$$

Back-substituting this value into C₁φ,s allows for an efficient 1D search over φ∈0,2π to be performed.

In another approach, Gaussian statistics for two populations of measured {Aᵢ} and {Bⱼ} are produced. {Aᵢ} and {(Bⱼ} naturally behave as Gaussian distributions as the amount of data becomes large. If, however, the population is not large enough, it is possible to ‘corral’ the data so that it has Gaussian statistics, using the following approach.

A Gaussian on SE(3) can be defined when the norm ∥Σ∥ is small as

$$ρ H ; M , \sum = \frac{1}{2 {π}^{3} \vert \sum \vert \frac{1}{2}} {e}^{- \frac{1}{2} F {M}^{- 1} H}$$

where |Σ| denotes the determinant of Σ and

FH=[ log [symbol] H]^{T}Σ⁻¹[ log [symbol] H].

When H is parameterized with exponential coordinates, H=expZ, this means that F expZ=z^{T}Σ⁻¹z where z=Z [symbol] and ρ expZ; [symbol] ₄,Σ becomes a zero-mean Gaussian distribution on the Lie algebra SE(3), with covariance Σ.

There is no loss in generality in assuming that ∥Σ_{A}∥ and ∥Σ_{B}∥ are small because the constraint equation (10) is linear in ΣA and Σ_{B}, and so if they are not small, they can be normalized resulting in Σ′_{A}=Σ_{A}/10∥Σ_{A}∥ and likewise Σ′_{B}=Σ_{B}/10∥Σ_{B}∥.

Moreover, standard tests from multivariate statistical analysis such as q-q plots can be used to assess whether or not the data sets are Gaussian. If not, they can be made Gaussian without loss of information or by introducing changes to the original mean and covariance. Since Aᵢ=XBᵢX⁻¹, it follows that Aᵢ^{p}=XBᵢ^{p}X⁻¹ for any power p∈ [symbol] . Practically speaking, we can sample p at fractional powers in the range p∈−1,1 and introduce multiple instances of samples with a Gaussian weighting that depends on p, causing the resulting augmented data set to behave as a Gaussian.

Solving for X can therefore be reduced to finding the global minimum of the cost function

C₂X=D_{KL}ƒ_{A}∥δ_{X}*ƒ_{B}*δ_{X-1} (19)

where ƒ_{A}H=ρH; M_{A},ΣA and

δ_{X}*ƒ_{B}*δ_{X-1}H=ρH;XM_{B}X⁻¹,AdXΣ_{B}Ad^{T}X.

In general, the integral in this cost function cannot be solved in closed form because the log function is nonlinear, and in terms of exponential coordinates dH=|J(z)|dz where |J0|=1, but this Jacobian is a nonlinear function of z.

However, if a priori we limit the search for X to the cylinder defined in (17), then XM_{B}X⁻¹=M_{A}. We can then define a variable K=M_{A}⁻¹H, and using the property of invariance of integration under shifts,

C₂Xφ,s=D_{KL}ƒ′_{A}∥ƒ′_{B}

where

ƒ′_{A}(K)=ρK; [symbol] ₄,Σ_{A}

and

ƒ′_{B}(K)=ρK; [symbol] ₄,AdXφ,sΣ_{B}Ad^{T}Xφ,s.

When restricting to the cylinder, logarithms and exponentials cancel. Moreover, scaling covariances so that they are small ensures that the integral over SE(3) reduces to a Gaussian integral over se(3)≅ [symbol] ⁶ since |J0|=1. The KL divergence of two distributions on [symbol] ⁿ with means mᵢ and covariances Σᵢ is

$${D}_{KL} {f}_{1} \vert \vert {f}_{2} = \frac{1}{2} [ tr {\sum }_{2}^{- 1} {\sum }_{1} + {m}_{2} - {{m}_{1}}^{T} {\sum }_{2}^{- 1} {m}_{2} - {m}_{1} - n - ln ( \frac{\vert {\sum }_{1} \vert}{\vert {\sum }_{2} \vert} ) ] .$$

In our problem, m₂−m₁=0 and since SE(3) is unimodular, |Ad X|=1 and so when evaluating Σ₁=ΣA and Ad X φ,s Σ_{B}Ad^{T} X φ,s, the final term in the above expression for D_{KL} ƒ₁∥ƒ₂ is independent of X. Moreover, for our purposes additive and positive multiplicative constants can be ignored and so we can simply consider the first term in the KL-divergence, scaled by a factor of two:

C′₂Xφ,s=trΣ_{A}⁻¹AdXφ,sΣ_{B}Ad^{T}Xφ,s,

minimized over φ,s∈0,2π× [symbol] . Minimization over s can be done in closed form since [symbol] C′₂ X φ,s is also quadratic in s, and the result is substituted back in for a one-dimensional search over Ø.

The preceding methods assume that the data are free of measurement errors. It is possible, however, to modify these approaches to allow for the fact that noise may exist in the data.

If X*=X φ*,s* is the optimal solution computed using any of the above methods, and this is treated as a good initial estimate rather than the final solution when the data have noise, then the optimal solution with noise can be written as X=X−X*Y where ∥log Y∥ is small. Then, without assuming that M_{A}=XM_{B}X⁻¹, but assuming that ∥Σ_{A}∥ and ∥log M_{A}∥ are small (and likewise for B), then

$${log}^{} {M}_{B}^{- 1} H \approx [ 𝕀 + \frac{1}{2} Ad {M}_{B} ] {log}^{} H$$

$$and$$

$$Ad X * Y = Ad X * Ad Y = Ad X * [ {𝕀}_{6} + ad log Y ]$$

$$where$$

$$ad log Y = [ \begin{matrix}Ω & v \\ 0 & 0\end{matrix} ] so ad ( log Y ) = [ \begin{matrix}Ω & 𝕆 \\ V & Ω\end{matrix} ]$$

and has the property that exp ad log Y=Ad expY.

An exact solution to the AⁱX=XBⁱ problem only exists when four independent invariant quantities exist between two pairs of A's and B's, which are defined from classical screw theory. From screw theory it is known that any homogenous transformation can be written as

$$H = ( \begin{matrix}{e}^{θ N} & ( {𝕀}_{3} - {e}^{θ N} ) p + d n \\ {0}^{T} & 1\end{matrix} )$$

where e^{θN} denotes the matrix exponential, [symbol] ₙ is the n×n identity matrix, and θΣ0, π is the angle of rotation.

$$N = ( \begin{matrix}0 & - {n}_{3} & {n}_{2} \\ {n}_{3} & 0 & - {n}_{1} \\ - {n}_{2} & {n}_{1} & 0\end{matrix} )$$

where n=[n₁, n₂, n₃]^{T}∈ [symbol] ³ is the unit vector describing the axis of rotation, which connects the origin and any point on the unit sphere, and p·n=0. Together, {θ, d, n, p} define the Plücker coordinates of the screw motion.

If we write AⁱX=XBⁱ as

Aⁱ=XBⁱX⁻¹ where i∈{1,2}, (20)

then calculating and equating the matrix product gives two invariant relations,

θ_{A}_{ⁱ}=θ_{B}_{ⁱ} d_{A}_{ⁱ}=d_{B}_{ⁱ}, (21)

where d_{A}_{ⁱ} and d_{B}_{ⁱ} are computed from Aⁱ and Bⁱ. Additionally, let

1_{A}_{ⁱ},(t)=p_{A}_{ⁱ}+tn_{A}_{ⁱ} and 1_{B}_{ⁱ},(t)=p_{B}_{ⁱ}+tn_{B}_{ⁱ}

be the directed screw axis lines of Aⁱ and Bⁱ in three-dimensional Euclidean space. If the lines are not parallel or anti-parallel, i.e. if n_{A}_{ⁱ}≠±n_{B}_{ⁱ}, then the distance between the two lines is given by

$$Δ ( {1}_{{A}^{i} 1} , {1}_{{A}^{i} 2} ) = \frac{‖ {n}_{{A}^{i} 1} , {n}_{{A}^{i} 2} , {p}_{{A}^{i} 2} - {p}_{{A}^{i} 1} ‖}{‖ {n}_{{A}^{i} 1} \times {n}_{{A}^{i} 2} ‖} \qquad ( 22 )$$

where for any a,b,c∈ [symbol] ³ the triple product is a,b,c≐a·b×c.

If in addition, Δ(1_{A}_{ⁱ}₁,1_{A}_{ⁱ}₂)≠0, i.e., if the lines are skew, then the angle φ(1_{A}_{ⁱ}₁,1_{A}_{ⁱ}₂)∈0,2π is uniquely specified by

cos φ(1_{A}_{ⁱ}₁,1_{A}_{ⁱ}₂)=n_{A}_{ⁱ}₁·n_{A}_{ⁱ}₂

sin φ(1_{A}_{ⁱ}₁,1_{A}_{ⁱ}₂)=Δ(1_{A}_{ⁱ}₁,1_{A}_{ⁱ}₂)⁻¹[n_{A}_{ⁱ}₁,n_{A}_{ⁱ}₂,p_{A}_{ⁱ}₂−p_{A}_{ⁱ}₁] (23)

Therefore, if (θ_{A}_{ⁱ}₁,θ_{A}_{ⁱ}₂)∈(0,π) and φ(1_{A}_{ⁱ}₁,1_{A}_{ⁱ}₂)∉{0,π}, then a unique solution of AⁱX=XBⁱ exists if and only if the following four conditions hold:

θ_{A}_{ⁱ}₁,θ_{B}_{ⁱ}₁ and θ_{A}_{ⁱ}₂,θ_{B}_{ⁱ}₂; 1)

d_{A}_{ⁱ}₁,d_{B}_{ⁱ}₁ and d_{A}_{ⁱ}₂,d_{B}_{ⁱ}₂; 2)

φ(1_{A}_{ⁱ}₁,1_{B}_{ⁱ}₂)=φ(1_{B}_{ⁱ}₁,1_{B}_{ⁱ}₂) 3)

Δ(1_{A}_{ⁱ}₁,1_{B}_{ⁱ}₂)=Δ(1_{B}_{ⁱ}₁,1_{B}_{ⁱ}₂) 4)

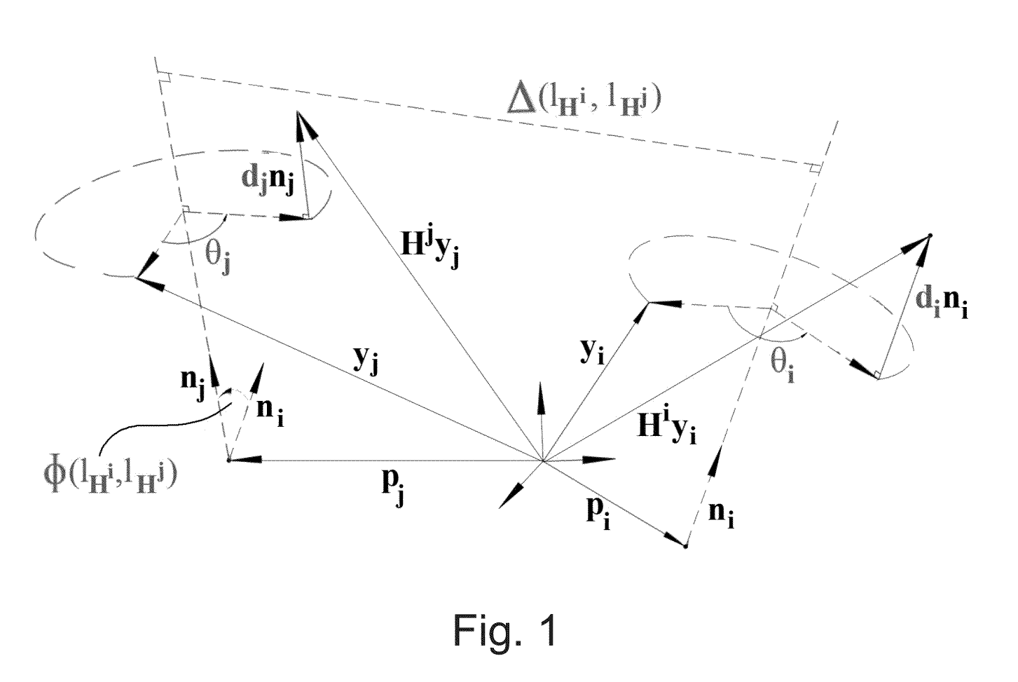

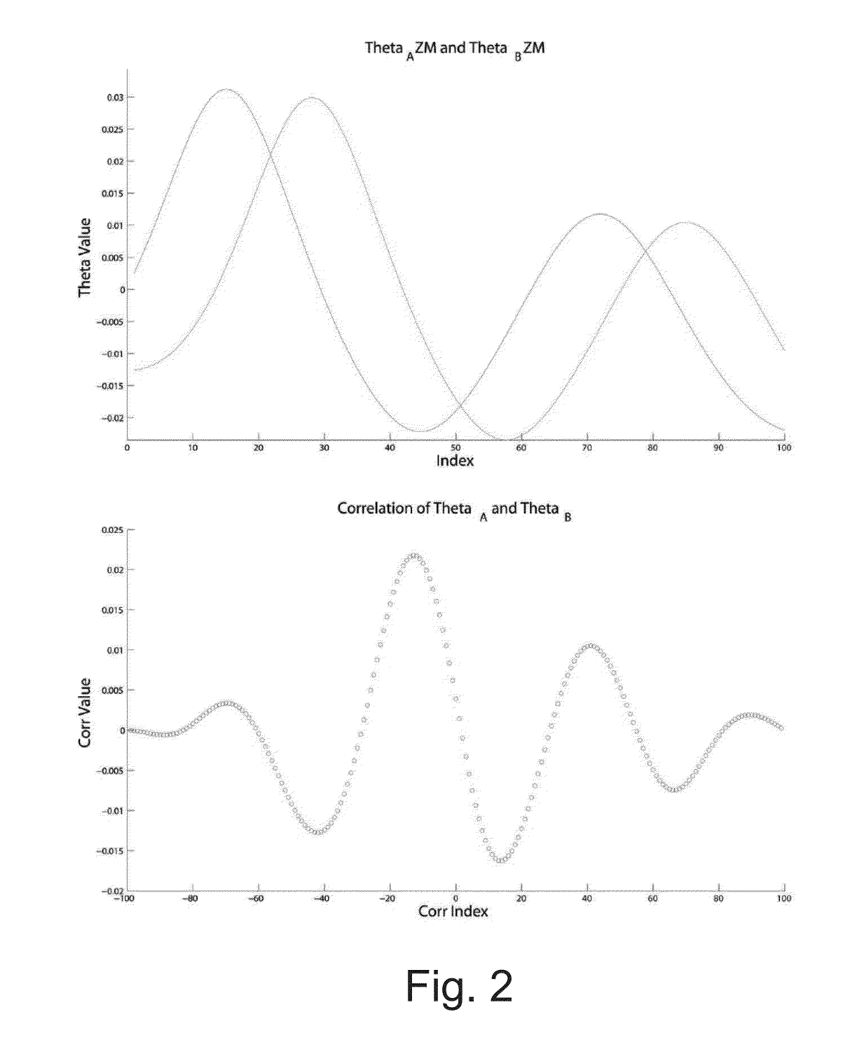

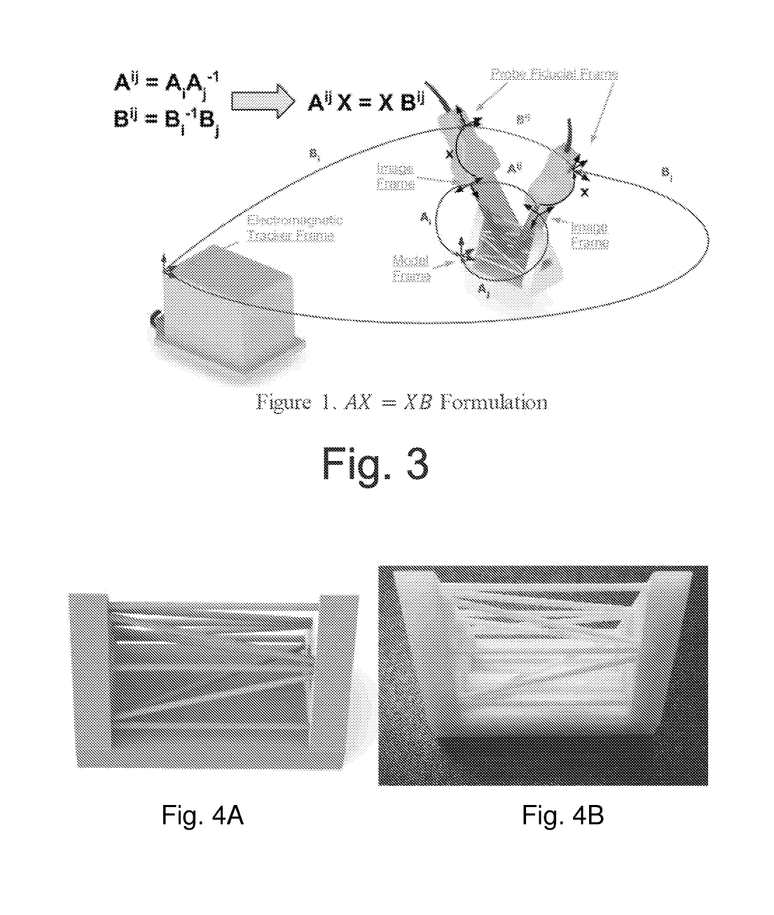

FIG. 1 illustrates the Plücker coordinates, and the parameters of the above four conditions for two arbitrary rigid-body motions.

One method of solving for X uses the first two invariants, θ and d, to compute a correlation by re-shifting temporally misaligned data. Given n, (Aᵢ, Bᵢ) pairs drawn from sensor data, the set of A's is shifted by a set amount to mimic the effects of the asynchronous data transfer. The SE(3) invariants are then extracted from each of the Aᵢ's and Bᵢ's in the new temporally shifted set. We can then perform a correlation of the A invariants (θ_{A}, d_{A}) with the B invariants (θ_{B}, d_{B}) using the Discrete Fourier Transform.

Given a sequence of N complex numbers, the DFT is defined as

$${F}_{n} = ℱ ⌊ {\{ {f}_{k} \}}_{k = 0}^{N - 1} ⌋ ( n ) where {F}_{n} ≐ {\sum }_{k = 0}^{N - 1} {f}_{k} {e}^{- 2 π in \frac{k}{N}}$$

and the inverse transform is given as

$${f}_{k} ≐ \frac{1}{N} {\sum }_{k = 0}^{N - 1} {F}_{n} {e}^{2 π ik \frac{n}{N}} .$$

The convolution theorem for the Discrete Fourier Transform indicates that a correlation, Corr ƒ,g, of two sequences of finite length can be obtained as the inverse transform of the product of one individual transform with the complex conjugate (*) of the other transform:

Corr ƒ,g= [symbol] ⁻¹F·G*.

The location of largest correlation corresponds with the amount of shift in the A's. FIG. 2 shows an example case where the data streams are shifted by −13 units. The shifted theta streams can be seen in the top graph and the correlated plot, where the maximum value is at the predicted location (−13), is shown in the bottom graph. This method accurately recovers the shift in the data streams and then corrects the shift to calculate for X.

This approach is accurate with uniform shifts, or if the data has large gaps that allow for substreams to be created between gaps. However, if there are largely varying, non-uniform shifts, or a large number of small groups in the data, the previous algorithm begins to break down. As an alternative option, an algorithm using all four invariants is presented.

Consider the case that data streams of sensor measurements | [symbol] |={Aⁱ} and | [symbol] B|={Bⁱ} are presented with significant unknown temporal shifts between the two sets, and gaps within each one. The number of points in these sets are | [symbol] |=m and | [symbol] |=n.

Here we present an approach to recovering X and establishing a correspondence between the subsets [symbol] ′ ⊂ [symbol] and [symbol] ′⊂ [symbol] that do correspond where | [symbol] ′|=| [symbol] ′|=p≦min(m,n). For such data, we find the correspondence, which is a permutation on p letters, π∈Πₚ, such that AⁱX=XB^{π(i)} for i=1, . . . , p where Aⁱ∈ [symbol] ′ and B^{π(i)}∈ [symbol] ′.

This is accomplished using the invariants of the Special Euclidean group, SE(3), under conjugation. The procedure is as follows. Compute (θ^{A}^{^{I}},d^{A}^{ⁱ}) for each Aⁱ∈ [symbol] and (θ_{B}_{^{j}},θ_{B}_{^{j}}) for each B^{j}∈ [symbol] . Next, form a 2D grid on the θ-d plane that ranges from min_{i,j}(θ_{A}_{ⁱ},θ_{B}_{^{j}}) to max_{i,j}(θ_{A}_{ⁱ},θ_{B}_{^{j}}) and min_{i,j}(d_{A}_{ⁱ},d_{B}_{^{j}}) to max_{i,j}(d_{A}_{ⁱ},d_{B}_{^{j}}). This grid will give r rectangles, e.g., if it is a 10×10 grid, then r=100. Assuming that no data falls exactly on a grid line, this will partition A and B into r disjoint subsets: [symbol] ₁, [symbol] ₂, . . . , [symbol] ᵣ and [symbol] ₁, [symbol] ₂, . . . , [symbol] ᵣ where

$${A}_{{i}_{1}} ⋂ {A}_{{i}_{2}} = 0 / and {⋃}_{i = 1} {A}_{i} = A ,$$

and similarly for B.

This allows for all potentially matching A's and B's to be in corresponding partitions Aᵢ and Bᵢ, since having the same value of θ and d is a necessary condition for a solution to AX=XB to exist. Constructing the grid with finite resolution allows for the possibility of some measurement errors in A's and B's.

Let | [symbol] ᵢ|=mᵢ and | [symbol] ⱼ|=nⱼ, then

$${\sum }_{i = 1}^{r} {m}_{i} = m and {\sum }_{i = 1}^{r} {n}_{j} = n .$$

Pick two bins for which all of the numbers in the pairs (mᵢ_{₁},nⱼ_{₁}) and (mᵢ_{₂},nⱼ_{₂}) are small, but greater than 2, to allow for the fact that measurement error may result in incorrect binning, and also that the angle φ(1_{A}_{ⁱ}₁,1_{A}_{ⁱ}₂) might not always be in the range (0,π). We interrogate all mᵢ_{₁}×nⱼ_{₁}×mᵢ_{₂}×nⱼ_{₂} possibilities as candidates. The further necessary conditions for the existence of a solution are φ(1_{A}_{ⁱ}₁,1_{A}_{ⁱ}₂)=φ(1_{B}_{^{j}}₁,1_{B}_{^{j}}₂) and Δ(1_{A}_{ⁱ}₁,1_{A}_{ⁱ}₂)=Δ(1_{B}_{^{j}}₁,1_{B}_{^{j}}₂). From among all pairs that satisfy these conditions, we can use existing AX=XB solvers to determine X.

To solve the, now registered, AX=XB, a common method that finds a least-squares solution is used by using the Kronecker product. If C is a matrix, vec(C) is the long vector produced by stacking the columns of C. This is a linear operation in the sense that vec α₁C₁+α₂C₂=α₁vec C₁+α₂vec C₂. Moreover, if [symbol] denotes the Kronecker product, and C, D, E are matrices with dimensions compatible for multiplication, then vec CDE=E^{T} [symbol] C vec D. If D is already a column vector (n×1 matrix), then it is unaltered by the vec(•), and if D is a row vector (1×n matrix), then vec(•) transposes it. If C is a matrix and α is a scalar, then α [symbol] C=C [symbol] α=αC, the scalar multiple of C by α.

Therefore, we can write the AX=XB equation as

$$\begin{matrix}{J}_{i} x = {b}_{i} & \\ where & \\ {J}_{i} = [ \begin{matrix}{𝕀}_{9} - {R}_{{B}_{i}} ⊗ {R}_{{A}_{i}} & {𝕆}_{9 \times 3} \\ {t}_{{B}_{i}}^{T} ⊗ {𝕀}_{3} & {𝕀}_{3} - {R}_{{A}_{i}}\end{matrix} ] , & ( 24 ) \\ x = [ \begin{matrix}vec {K}_{{R}_{X}} \\ {K}_{{t}_{X}}\end{matrix} ] and {b}_{i} = [ \begin{matrix}{0}_{9} \\ {t}_{{A}_{i}}\end{matrix} ] , & ( 25 )\end{matrix}$$

[symbol]

ₘ is the m×m identity, [symbol] _{m×n} is the m×n zero matrix, and 0ₙ is the n-dimensional zero vector.

Jᵢ is a 12×6 matrix and bᵢ is six-dimensional. By stacking multiple such equations for different pairs, (Aᵢ, Bᵢ), we obtain Jx=b where J is 12n×6n and b is 6n-dimensional. The least-squares solution for x can be then found using SVD methods or using a pseudo-inverse.

For example, the least-squares solution to ∥Jx−b∥_{M} where M=M^{T}∈ [symbol] ^{6n×6n} is

x=J^{T}MJ⁻¹J^{T}Mb (26)

This is the over-constrained pseudo-inverse, as opposed to the under-constrained pseudo-inverse typically used in redundancy resolution.

For some applications of the AX=XB problem, the sensor data is only rotational. The (Aᵢ, Bᵢ) pairs are now drawn form the group of rigid-body rotations, SO(3), a subgroup of SE(3). In this case there is no d or Δ. The presented algorithms are successful for data of this type, despite the absence of translation information. For the algorithm using correlations, θ is used to match the data streams. The second algorithm, which uses the binning procedure, is successful using only the θ and Ø invariants.

It is also possible to solve for X in the AX=XB problem using a Batch Method, the algorithm as follows. SE(3) is a Lie group, and hence concepts of integration and convolution exist. If X∈SE(3) is a generic 4×4 homogeneous transformation of the form H R,

$$t = ( \begin{matrix}R & t \\ {0}^{T} & 1\end{matrix} )$$

where the rotation is parameterized in terms of Euler angles as R=R₃ α R₁ β R₃ γ (where Rᵢ(θ) is a counterclockwise rotation by θ around coordinate axis i) and the translation is t=[tₓ, t_{y},t_{z}]^{T}, then the natural integral of any rapidly decaying function is computed as

∫_{SE(3)}ƒHdH= [symbol] ³∫_{SO(3)}ƒHR,tdRdt

where dR=sin βdαdβdγ and dt=dtₓdt_{y}dt_{z}, with −∞<tₓ,t_{y},t_{z}<∞ and |α,β,γ|∈0,2π×0, π×0,2π. This integral is natural in the sense that it is unique such that

∫_{SE(3)}ƒHdH=∫_{SE(3)}ƒH⁻¹dH=∫_{SE(3)}ƒHH₀dH=∫_{SE(3)}ƒH₀HdH (27)

for any fixed H₀∈SE(3). This choice of integral, being invariant under shifts on the left and on the right in the above equation, is called the bi-invariant measure. This instantiation of the bi-invariant integral for SE(3) is not unique, as any parameterization of SE(3) can be used.

If ∫_{SE(3)}|ƒH|^{p}dH<∞ then we say ƒ∈L^{p}(SE(3)). Most of the following algorithm will be limited to functions ƒ∈L¹(SE(3))∩L²(SE(3)), together with the special case of a Dirac delta function.

In this light, we can think of AᵢX=XBᵢ as the equation

$$( {δ}_{{A}_{i}} * {δ}_{x} ) H = ( {δ}_{x} * {δ}_{{B}_{i}} ) H \qquad ( 28 )$$

This provides freedom to execute mathematical operations that could not be previously be performed. The addition of homogeneous transformation matrices is nonsensical, but can be performed with real-valued functions.

In this form, the correspondence between Aᵢ and Bⱼ does not need to be known, as the above functions are normalized to be probability densities:

∫_{SE(3)}ƒ_{A}HdH=∫_{SE(3)}ƒ_{B}HdH=1.

Assume that the set of Aᵢ's and Bᵢ's are clumped closely together. In other words, given a measure of distance between reference frames, d:SE(3)×SE(3)→ [symbol] _{≦0}, we have that d Aᵢ,Aⱼ, d Bᵢ,Bⱼ<∈<<1. This assumption can be made true, for example, if we are using small relative motions between consecutive reference frames, regardless of the length of the whole trajectory. The mean and covariance of a probability density ƒ(H) can be defined by the conditions

∫_{SE(3)} log M⁻¹HƒHdh=

[symbol]

and

Σ∫_{SE(3)} log [symbol] M⁻¹H[ log [symbol] M⁻¹H]^{T}ƒHdh (29)

The definition of mean used above differs from that most often used in literature when taking a Riemannian-geometric (rather than Lie-group) approach which is of the form M′=argmin_{M}∫_{SE(3)}[d M,H]² ƒ H dH where d(M,H) is A Riemannian distance function (d M,H=∥log M⁻¹,H∥_{W}², for example) and W is a weighting matrix related to the Riemannian metric tensor that is chosen. There are two reasons for our definition. First, in our definition there is no need to introduce a weighting matrix, and therefore we avoid coloring the result by arbitrary choice. Second, in the context of robotics problems in which reference frames are attached to rigid links it is more natural in the following sense.

If a single rigid link has a world frame attached to its base, and a reference frame attached to its distal end, and the distal reference frame is recorded at two different times as a joint at the base rotates, then the translation part of the average of these two reference frames should lie on the arc that joins the two. M will have this property, but M′ will not. If we consider data on SO(3) rather than SE(3), and if W= [symbol] were chosen, the two definitions would become the same thing since for SO(3) the distance function d R₁,R₂≐∥log R₁⁻¹R is bi-invariant and Ad(R)=R. But for SE(3) neither of these statements are true: Ad H≠H and there does not exist a bi-invariant metric.

If ƒ(H) is of the form ƒ_{A}(H) then

$${\sum }_{i = 1}^{n} log {M}_{A}^{- 1} {A}_{i} = 𝕆 and {\sum }_{A} = \frac{1}{n} {\sum }_{i = 1}^{n} {log}^{} {M}_{A}^{- 1} {{A}_{i} [ {log}^{} {M}_{A}^{- 1} {A}_{i} ]}^{T} \qquad ( 30 )$$

An iterative process for computing M_{A} is performed in which an initial estimate of the form

$${M}_{A}^{0} = exp ( \frac{1}{n} {\sum }_{i = 1}^{n} log {A}_{i} )$$

is chosen, and then a gradient descent procedure is used to update so as to minimize the cost C(M)=∥Σᵢ₌₁ⁿ log M⁻¹Aᵢ∥², and the minimum defines M_{A}.

It can be shown that if these quantities are computed for two highly focused functions, ƒ₁ and ƒ₂, that the same quantities for the convolution of these functions can be computed as

$${M}_{{1}^{*} 2} = {M}_{1} {M}_{2} and {\sum }_{{1}^{*} 2} = Ad {M}_{2}^{- 1} {\sum }_{1} {Ad}^{T} {M}_{2}^{- 1} + {\sum }_{2} \\ where Ad H = ( \begin{matrix}R & 𝕆 \\ tR & R\end{matrix} ) . \qquad ( 31 )$$

Here for any a∈ [symbol] ³, â is the skew-symmetric matrix such that âb=a×b. [symbol] is used as the reverse map which gives â [symbol] =a. The mean of δ_{X}H is M_{X}=X, and its covariance is the zero matrix. Therefore

(a) M_{A}X=XM_{B} and (b)AdX⁻¹Σ_{A}Ad^{T}X⁻¹=Σ_{B} (32)

These two equations can be solved in a similar way to how the equations A₁X=XB₁ and A₂X=XB₂ are solved.

First, we seek the rotational component, R_{X} of X. From (33a) we have that,

n_{M}_{_{A}}=R_{X}n_{M}_{_{A}} (33)

where n_{H} is the direction of the screw axis of the homogeneous transfer H.

If we decompose Σ_{M}_{_{A}} and Σ_{M}_{_{B}} into blocks as

$${\sum }_{i} = ( \begin{matrix}{\sum }_{i}^{1} & {\sum }_{i}^{2} \\ {\sum }_{i}^{3} & {\sum }_{i}^{4}\end{matrix} )$$

where Σᵢ³=Σᵢ²^{^{T}}, then we can take the first two blocks of (33b) and write

Σ_{M}_{_{B}}¹=R_{X}^{T}Σ_{M}_{_{A}}¹R_{X} and Σ_{M}_{_{B}}¹=R_{X}^{T}Σ_{M}_{_{A}}¹R_{X}R_{X}_{^{t}}ₓ^{T}+R_{X}^{T}Σ_{M}_{_{A}}²R_{X} (34)

We can then find the eigenvalue decomposition, Σᵢ=QᵢΛQᵢ^{T}, where Qᵢ is the square matrix whose ith column is the eigenvector of Σᵢ and A is the diagonal matrix with corresponding eigenvalues as diagonal entries and write the first block equation of (35) as,

A=Q_{M}_{_{B}}^{T}R_{X}^{T}Q_{M}_{_{A}}ΛQ_{M}_{_{A}}^{T}R_{X}Q_{M}_{_{B}}= [symbol] Λ [symbol] ^{T} (35)

The set of Qs that satisfy this equation is given as,

$$Q = \{ ( \begin{matrix}1 & 0 & 0 \\ 0 & 1 & 0 \\ 0 & 0 & 1\end{matrix} ) , ( \begin{matrix}- 1 & 0 & 0 \\ 0 & - 1 & 0 \\ 0 & 0 & 1\end{matrix} ) , ( \begin{matrix}- 1 & 0 & 0 \\ 0 & 1 & 0 \\ 0 & 0 & - 1\end{matrix} ) , ( \begin{matrix}1 & 0 & 0 \\ 0 & - 1 & 0 \\ 0 & 0 & - 1\end{matrix} ) \}$$

with the simple condition that Qᵢ is constrained to be a rotation matrix. This means that the rotation component of X is given by,

Rₓ=Q_{M}_{_{A}} [symbol] Q_{M}_{_{B}}^{T} (36)

The correct solution, form the set of 4 possibilities of Rₓ can be found by applying (34) and choosing the one that minimizes ∥n_{M}_{_{A}}−R_{X}n_{M}_{_{A}}∥. Once Rₓ is found in this way, tₓ can be found from blocks 2 and 4 of (11b). Through numerical experimentation, it can be seen that, unlike traditional least-squares solution methods using the Kronecker product which degenerate with slight permutations of As and Bs, the Batch Solver finds the correct solution with any amount of permutation.

The aforementioned algorithms can be used in sensor calibration for cameras, ultrasound probes, optical or magnetic pose tracking systems, and the like. The calibration of an ultrasound probe is discussed as a non-limiting example. This process recovers the rigid body transformation between a tracked marker attached to the ultrasound transducer and the ultrasound image. In order to perform this calibration, a phantom or model with known geometry is used.

Generally, the present invention approaches the design of this phantom with consideration of ultrasound physics, and ensuring that the calibration process is easy for the user. The AX=XB is also referred to as the hand-eye calibration problem. As seen in FIG. 3, Aⁱ and Bⁱ are relative motions connected by the rigid body transformation X. A tracker provides the homogeneous transformation Bⁱ. The present invention, which can be seen in FIG. 4, provides an AX=XB phantom through which the transformations, Aᵢ, relating each image to the phantom's coordinate system, can be computed. The AX=XB framework is advantageous, as it does not require the transducer to be fixed at a specific location, or for the calibration phantom to be tracked by the external tracker. In addition, the phantom can be created without any need for additional assembly, adjustment, or treatment. In contrast to designs where only the frame is manufactured from a three-dimensional printer, the present invention is printed in its entirety including the wires (also referred to as “rods”). The rods shown in FIG. 4 are 3-Dimensional rods with a circular cross section, however the rods can also have a cross section that is triangular, rectangular, hexagonal, and the like. This allows for the phantom to be “plug and play”—the user can download an appropriate computer-aided design (CAD) file, print it from a three-dimensional printer, and have the ability to perform ultrasound calibration.

The length and the angle of the Z-fiducials allow for submillimeter translation. Ultrasound physics define an axial dimension that has higher resolution than the lateral dimension. With varying orientations, a proportion of the Z-fiducial change is reflected in the axial dimension. However, rods that are not perpendicular to the imaging plane have a non-optimal acoustic response. Therefore, the geometric configuration of the present invention is designed to have rods in non-parallel planes such that when one Z-fiducial becomes difficult to visualize, another Z-fiducial will have a rod that becomes increasingly perpendicular to the image plane. In one non-limiting example, the planes may diverge from parallel in a range from −30 to 30 degrees. Furthermore, the phantom has redundant rods, such that the subset of rods with the highest acoustic response, given the imaging orientation, can be chosen.

Additionally, the phantom is configured to avoid the situation where many of the rods are shadowed. The Z-fiducials are oriented in the shape of a triangle such that when one face of the triangle is experiencing severe shadowing effects, the other faces will be unaffected. Furthermore, the phantom is designed such that probes of multiple lateral lengths can be used within the same phantom. Finally, the phantom allows for large range of motion of at least 3 cm for each translational degree of freedom and 45 degrees for each rotational degree of freedom.

The first step of the segmentation algorithm applies an intensity threshold to the image. A connected regions algorithm is the used to cluster signal pixels together. A filter is the applied where only regions containing a certain range of pixels are retained. These steps allow for removal of noise and extraction of the rods from the ultrasound image. Then, a region closest to the transducer face is selected, which corresponds to the top rod in the phantom. The remaining regions in the ultrasound image will exhibit a triangular shape. Thus, the standard Hough transform can be applied to find the edges of the triangular pattern. An understanding of the location of the top rod and the edges of the triangular pattern allows for the establishment of the correspondences between the triangular pattern and the model. For each region, points are selected which lie closest to the transducer face from the centroid, as the top of the rods are most accurately represented in ultrasound imaging, resulting with a localization that has better lateral and axial resolution.

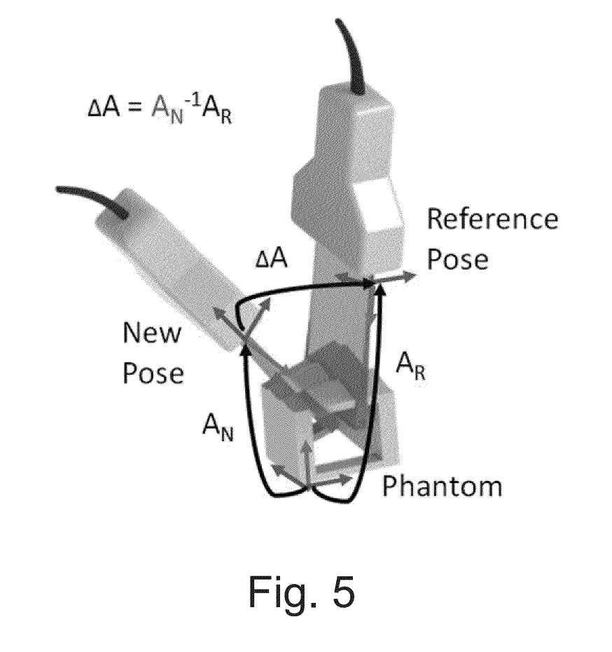

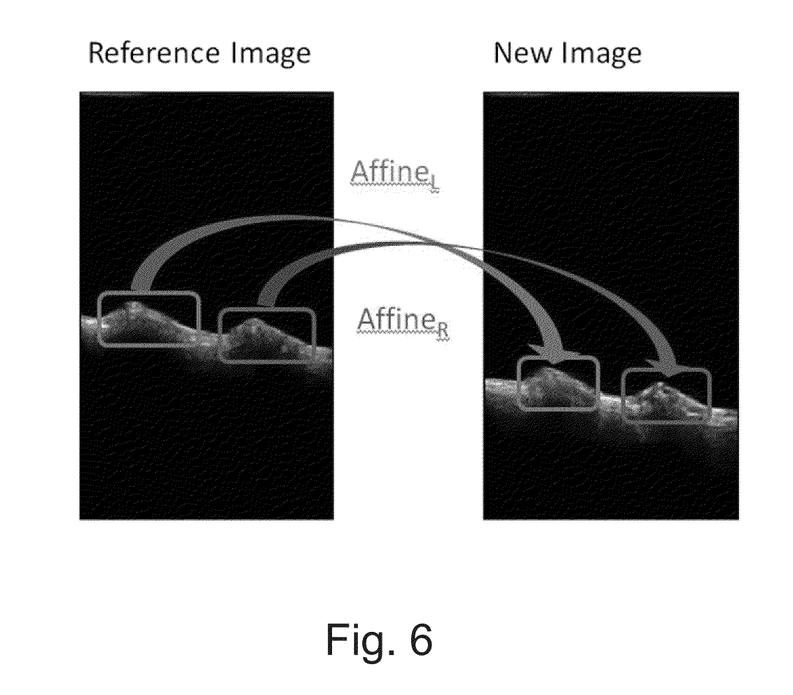

In another embodiment of the phantom, a double triangle pyramid phantom as shown in FIG. 5 is used. This phantom eliminates the rods previously described, and relies on points on the outer edge of a pyramid to perform calibration of the device. In this embodiment, a pose from a reference imaging system is used to create a reference image, and an additional pose from the imaging system to be calibrated is used to create a new image. The reference image and the new image are both composed of a left triangle and a right triangle, each triangle composed of three 3-dimensional points. As shown in FIG. 6, the known points in the reference image and the affine transformation of these points, determined with image registration and/or tracking methods, are used to determine corresponding points in the new image.

In a further application of this phantom, the reference pose is not used. When using this application during the calibration process, there are no longer six known points which correspond to the new image. With the increase in unknowns, more images are necessary to recover all of the poses.

The present invention has been described in terms of one or more preferred embodiments, and it should be appreciated that many equivalents, alternatives, variations, and modifications, aside from those expressly stated, are possible and within the scope of the invention.

1. A system for calibrating an imaging system comprising:
an input configured to receive data acquired by at least one sensor of an imaging system;
an input configured to receive data acquired by at least one sensor of a tracking system;
a processor configured to receive the data from the inputs and to:
use the data to populate a calibration model having a form of AX=BX, wherein A, X, and B are homogeneous transformations with A and B determined from the data and X being an unknown calibration parameter;
filter the data using screw theory invariants;
compute the calibration parameter, X, between A and B;
use solutions from Batch methods which do not require correspondence of the input data;
find correspondence using the invariants and a solution from a time-evolving method which allows real time computation and updates.

2. The system of claim 1 wherein the processor is configured to compute the calibration parameter, X, using a probabilistic Batch method that treats the data as probability density functions.

3. The system of claim 2 wherein the processor is configured to use mean and covariance to construct solvable, correspondence-free equations.

4. The system of claim 1 wherein the processor is configured to compute the calibration parameter, X, by formulating the data as a time-evolving differential equation.

5. The system of claim 4 wherein the processor is further configured to perform an online calibration of the imaging system using the correspondence between A and B.

6. The system of claim 5 further comprising a display configured to display an evolution of X with time.

7. The system of claim 5 wherein the screw theory invariants include θ, d, Δ and Ø, wherein:
θ is a rotational transformation about a fixed axis;
d is a translational transformation about the fixed axis;
Δ is a distance between a first directed screw axis line of a first three dimensional pose and a second directed screw axis line of a second three dimensional pose.
Ø is an angle between the first directed screw axis line and the second directed screw axis line.

8. The system of claim 5 where the imaging system to be calibrated includes ultrasound transducers, cameras, robot hands, optical pose tracking systems, and magnetic pose tracking systems.

9. A phantom for calibration of an imaging system, the phantom comprising:
a frame comprising:
first and second opposing walls;
a base with a first edge and a second edge, the first edge joined to an edge of the first opposing wall and the second edge joined to an edge of the second opposing wall; and
a plurality of rods spaced between the first and second opposing walls, wherein the rods lie in non-parallel planes.

10. The phantom of claim 9 wherein the phantom is manufactured entirely using a three-dimensional printer.

11. The phantom of claim 9 wherein the rods are oriented in a plurality of Z-fiducials with a skew from parallelism in the range of −30 to 30 degrees.

12. The phantom of claim 11 wherein the Z-fiducials are triangularly shaped.

13. The phantom of claim 9 wherein the phantom allows for a minimum of 3 centimeters translational movement and 45 degrees rotational movement of the imaging system to be calibrated.

14. A method for calibration of an imaging system, the method comprising the steps of:
providing a phantom;
placing an imaging system in a first position relative to the phantom;
acquiring a first ultrasound image of the phantom;
determining a spatial relationship between the phantom and the first image;
repositioning the imaging system in a second position relative to the phantom;
acquiring a second ultrasound image of the phantom;
determining a second spatial relationship between the phantom and the second image;
relaying the first and second spatial relationships to a processor; and
processing the spatial relationship data by:
using the data to populate a calibration model having a form of AX=BX, wherein A, X, and B are homogeneous transformations with A and B determined from the data and X being an unknown;
filtering the data using a screw theory;
disregarding portions of the data without desired screw theory invariants;
computing an unknown transformation between the imaging system and the phantom.

15. The method of claim 14, further comprising the step of obtaining spatial relationship data related to additional positions of the imaging system relative to the phantom.

16. The method of claim 14, wherein the spatial relationships between the phantom and the images are determined based on knowledge related to the location of the triangular Z-fiducials present in the image.

17. The method of claim 14, wherein the phantom comprises:
a frame comprising:
first and second opposing walls;
a base with a first edge and a second edge, the first edge joined to an edge of the first opposing wall and the second edge joined to an edge of the second opposing wall; and
a plurality of rods spaced between the first and second opposing walls, wherein the rods lie in non-parallel planes with a skew from parallelism in the range of −30 to 30 degrees.

18. A phantom for calibration of an imaging system, the phantom comprising:
first and second opposing walls;
a base with a first edge and a second edge, the first edge joined to an edge of the first opposing wall and the second edge joined to an edge of the second opposing wall;
a cover portion opposing the base, the cover portion having a first edge joined to an edge of the first opposing wall and a second edge joined to an edge of the second opposing wall; and
a plurality of apertures through the cover portion sized to receive a plurality of sensors.

19. The phantom of claim 18 wherein the plurality of sensors comprises a reference imaging system and the imaging system to be calibrated.
